(19) United States
(12) Patent Application Publication
(10) Pub. No.: US 2024/0180434 A1
(43) Pub. Date: Jun. 6, 2024

(54) SYSTEM AND METHOD FOR BLOOD PRESSURE MEASUREMENT, COMPUTER PROGRAM PRODUCT USING THE METHOD, AND COMPUTER-READABLE RECORDING MEDIUM THEREOF

(71) Applicant: Amengine Corporation, Zhubei City (TW)

(72) Inventors: Sheng-Chieh HUANG, Hsinchu (TW); Paul C.-P. CHAO, Taipei City (TW); Yung Hua KAO, Hsinchu (TW); Pei-Yu CHIANG, Hsinchu (TW)

(21) Appl. No.: 18/419,295

(22) Filed: Jan. 22, 2024

Related U.S. Application Data
(63) Continuation-in-part of application No. 17/242,906, filed on Apr. 28, 2021.
Application No. 17/242,906 is a continuation of application No. 15/204,392, filed on Jul. 7, 2016, now abandoned.

(30) Foreign Application Priority Data
Sep. 16, 2015 (TW) 104130568

(51) Int. Cl. A61B 5/021; A61B 5/00; A61B 5/024; G06N 3/08; G06N 3/086; G16H 10/60; G16H 50/50; G16H 50/70; G16Y 20/40; G16Y 40/10; H04L 67/12

(52) U.S. Cl. CPC A61B 5/02108; A61B 5/02125; A61B 5/02133; A61B 5/7278; G06N 3/08; G06N 3/086; G16H 50/50; G16H 50/70; A61B 5/024; A61B 5/681; A61B 5/6898; A61B 5/7264; G16H 10/60; G16Y 20/40; G16Y 40/10; H04L 67/12

(57) ABSTRACT

The present invention provides a system and method for blood pressure measurement, a computer program product using the method, and a computer-readable recording medium thereof. The present invention uses a sensor to measure an electrophysiological signal and establishes a personalized cardiovascular model through a numerical method, and re-establishes the personalized cardiovascular model through an optimization algorithm. Thus, a human physiological parameter generated from the re-established personal cardiovascular model matches the electrophysiological signal. Therefore, the present invention can provide accurate measurement results with the advantage of a small size, and can be applied to telemedicine field.

### RELATED APPLICATION DATA

This application is a continuation-in-part application of and claims the priority benefit of U.S. application Ser. No. 17/242,906 filed Apr. 28, 2021, now pending, which is a continuation of U.S. application Ser. No. 15/204,392 filed Jul. 7, 2016, now abandoned, which claims the right of priority of TW Application No. 104130568, filed on Sep. 16, 2015, the contents of which are hereby incorporated by reference in their entireties.

### BACKGROUND OF THE INVENTION

### 1. Field of the Invention

The present invention relates to blood pressure measurement, and, more particularly, to a system and method for blood pressure measurement that establishes a personalized cardiovascular model using a finite element method, a computer program product using the same, and a computer-readable recording medium thereof.

### 2. Description of Related Art

Due to changes in modern lifestyle and refined diet, there has been a substantial increase in various chronic diseases, such as hypertension, diabetes, high cholesterol or cardiovascular diseases and the like. In pursuit of health, there are more and more passive electronic physiological measuring instruments emerging on the market that allow people to monitor their health in their own homes, enabling convenient and rapid physiological measurements.

In terms of measuring the blood pressure, mercury sphygmomanometers and oscillometric blood pressure monitors, for example, are commercially available. Although mercury sphygmomanometers provide accurate blood pressure measurement results, it requires professional personnel or trained person to carry out the operation, otherwise it is difficult to obtain satisfactory measurements. As a result, users cannot monitor their own health at any time. Oscillometric blood pressure monitors are more convenient to carry and operate than mercury sphygmomanometers, but as they employ the principle of oscillation for detection, an oscillometric blood pressure monitor has to be bound to the arm via a cuff, and the tightness of the cuff will affect the magnitude of the blood pressure measured, leading to low accuracy in the monitoring results.

Therefore, there is a need for a blood pressure measuring technique that addresses the aforementioned issues in the prior art.

### SUMMARY OF THE INVENTION

In view of the aforementioned shortcomings of the prior art, a main objective of present invention is to provide a method for blood pressure measurement, which may include: measuring an electrophysiological signal and obtaining basic personal information; establishing a personalized cardiovascular model based on the electrophysiological signal and the basic personal information through a numerical method, wherein the personalized cardiovascular model includes default model parameters and is used to create a human physiological parameter; and calibrating the default model parameters through an optimization algorithm to re-establish the personalized cardiovascular model, such that the human physiological parameter created by the re-established personalized cardiovascular model matches the measured electrophysiological signal.

Another objective of present invention is to provide a system for blood pressure measurement, which may include: a measuring unit for measuring an electrophysiological signal and including a user interface allowing basic personal information to be input thereon; and a computing unit connected with the measuring unit via a network for receiving the electrophysiological signal and the basic personal information. The computing unit may include: a modeling module for establishing a personalized cardiovascular model based on the electrophysiological signal and the basic personal information through a numerical method, wherein the personalized cardiovascular model includes default model parameters and is used to create a human physiological parameter; and an optimization module for calibrating the default model parameters through an optimization algorithm to re-establish the personalized cardiovascular model, such that the human physiological parameter created by the re-established personalized cardiovascular model matches the measured electrophysiological signal.

Yet another objective of present invention is to provide a computer program product for executing the method for blood pressure measurement described previously after being loaded into a machine.

Still another objective of present invention is to provide a computer-readable recording media, within which a computer program is stored. The computer program, after being loaded into a machine, is configured to execute the method for blood pressure measurement described previously.

In the system and method for blood pressure measurement, the computer program product that uses the method, and the computer readable recording medium thereof in accordance with the present invention, the blood pressure measuring sensor used is in the form of a vibrating pulse sensor or an optical sensor. The present invention thus eliminates the inconvenience associated with traditional cuff-type blood pressure measuring devices. The present invention also has a smaller volume which makes it easier to carry. In addition, the present invention allows the establishment of the personalized cardiovascular model in the cloud server and the calibration of the personalized cardiovascular model through the optimization algorithm. As a result, the human physiological parameter created by the personalized cardiovascular model can be more accurate. Furthermore, the personalized cardiovascular model can be used in the telemedicine field, facilitating long-range medical care services and early warning systems.

### BRIEF DESCRIPTION OF THE DRAWINGS

The present invention can be more fully understood by reading the following detailed description of the preferred embodiments, with reference made to the accompanying drawings, wherein:

FIG. 1 is a flowchart illustrating the steps of a method for blood pressure measurement in accordance with the present invention;

FIG. 2 is a block diagram depicting a system for blood pressure measurement in accordance with the present invention;

FIG. 3 is a flowchart illustrating the steps of a method for blood pressure measurement in accordance with the present invention;

FIG. 4 is a schematic diagram of signal waveforms in different steps;

FIG. 5 is a schematic diagram of a characteristic signal;

FIG. 6 is a model architecture of the pre-trained model;

FIG. 7 is a schematic diagram of the process of 5-fold cross validation;

FIG. 8 is a model architecture of the fine-tuned model;

FIG. 9 is a flowchart illustrating the steps of a method for biometric identification in accordance with the present invention; and

FIG. 10 is a model architecture of two biometric identification models.

### DETAILED DESCRIPTION OF THE EMBODIMENTS

The present invention is described by the following specific embodiments. Those with ordinary skills in the arts can readily understand other advantages and functions of the present invention after reading the disclosure of this specification. The present disclosure may also be practiced or applied with other different implementations.

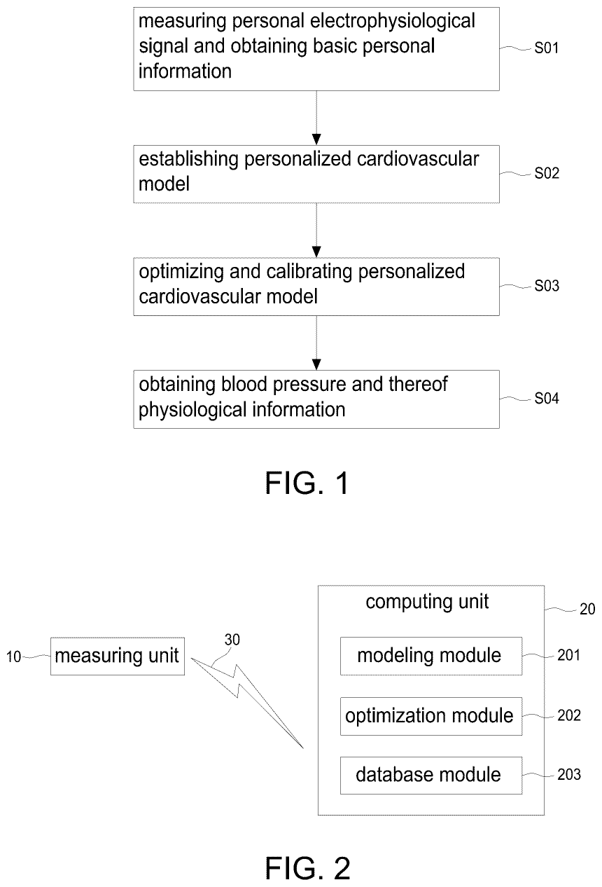

Referring to FIG. 1, a method for blood pressure measurement in accordance with the present invention includes: measuring an electrophysiological signal and obtaining basic personal information of an individual (step S01); establishing a personalized cardiovascular model (step S02); optimizing and calibrating the personalized cardiovascular model (step S03); and obtaining blood pressure and physiological information thereof (step S04).

In step S01, a personal electrophysiological signal is measured and basic personal information is obtained. More specifically, a pulse signal is measured by a sensor as the personal electrophysiological signal. The sensor may be a vibrating pulse sensor or an optical sensor.

In use, a vibrating pulse sensor is first attached to the wrist, and a pulse signal is acquired via a vibrating sensing unit in the sensor. An optical sensor can be a camera in a smartphone. During measurement, the camera obtains an optical signal that is not visible to the human eyes, and the optical signal is then converted into a personal pulse signal through an image processing algorithm.

In one embodiment, the vibrating pulse sensor can be a smartwatch. A pulse signal, after obtained, is wirelessly transmitted to a cloud server (e.g., infrared, Bluetooth, WiFi etc., but the present invention is not limited as such). Alternatively, the pulse signal is transmitted to a nearby smartphone first, and then transmitted to a cloud server via the smartphone. In one embodiment, the pulse signal may be transmitted to a nearby computer or a WiFi sharing device first, and then transmitted to a cloud server. The optical sensor may transmit the pulse signal to a cloud server directly from the smartphone.

Before or after the personal electrophysiological signal is measured, basic personal information is obtained via a user interface. In an embodiment, the basic personal information may include, but not limited to information such as age, height, weight, gender, body fat ratio etc., and/or personal information contained in the cloud electronic medical records. The user interface is a data input interface provided by an app in the smartwatch or the smartphone.

In step S02, after obtaining the personal electrophysiological signal and basic personal information, a personalized cardiovascular model is built by a numerical method. In an embodiment, the personalized cardiovascular model has default model parameters, and creates a human physiological parameter. In establishing the personalized cardiovascular model, based on the basic personal information of the individual, parameters such as vascular wall thickness, vessel diameter, vessel density, blood vessel elasticity, blood concentration, blood density, blood viscosity coefficient, blood flow, blood flow field pressure, muscle elasticity and density, elasticity and density of the bones, skin elasticity, or elasticity and density of all the tissues from the wrist continuously extended to the heart are set as the default model parameters of the personalized cardiovascular model. These default model parameters will have different default values depending on the parameters such as age, height, weight and gender. Based on these default model parameters, a personalized cardiovascular model can be built by a numerical method, e.g., the finite element method. In one embodiment, the personalized cardiovascular model is an arterial finite element model, and is built from the measuring point (i.e., the wrist).

In one embodiment, the personalized cardiovascular model may be further based on a new Blood Pressures Transport Theory (BPTT) modeled from the wrist continuously extending to the heart, and its model parameters are calibrated according to the size and direction of the pulse wave vector in the measured pulse signal. This ultimately becomes the personalized cardiovascular finite element model. The personalized cardiovascular model can be formed into a personalized radial artery cardiovascular finite element model.

After the personalized cardiovascular model is acquired, the human physiological parameter of blood pressure (BP) can be obtained from the following formula:

$$BP = C ( \frac{1}{2} ρ \frac{{d}^{2}}{{PTT}^{2}} + ρ gh ) ,$$

wherein C is the cardiovascular constant, ρ the blood density, d the blood vessel distance between the heart and the measuring point, PTT the average velocity of blood from the heart to the measuring point, g the gravitational constant, and h the height difference between the heart and measuring point.

In step S03, as the personalized cardiovascular model is initially built based on the default values of the model parameters that correspond to the basic personal information (such as age, height, weight, gender, body fat ratio, or data contained in the cloud electronic medical records etc.), it may not accurately reflect the individual status using the same default values, since persons have different cardiovascular status in reality, the default model parameters are calibrated using an optimization algorithm so the personalized cardiovascular model can better match the personal electrophysiological signal measured.

In one embodiment, the optimization algorithm is a genetic algorithm, a neural network algorithm, an intelligent algorithm, or any novel algorithm that automatically optimizes and calibrates the default values of the model parameters using a large number of samples.

In another embodiment, physiological information, such as the systolic blood pressure, the diastolic blood pressure, or the heart rate obtained from, for example, health checks or other blood pressure measuring devices can be used as the basis for calibrating the electrophysiological signal, and a more accurate formula for calculating blood pressure can be formed through the self-calibrating optimization algorithm as described before.

In step S04, based on the adjusted model parameters, the personalized cardiovascular model is re-established, such that the human physiological parameter created from the re-established personalized cardiovascular model matches the electrophysiological signal of the individual, and measurement results of the physiological information (e.g., the systolic blood pressure, the diastolic blood pressure or the heart rate) can be obtained.

The present invention also provides a computer program product for executing the method for blood pressure measurement previously described after being loaded into a machine (such as a computer or a smart phone). The present invention further provides a computer-readable recording media, such as a CD, a DVD, a flash drive, a hard drive, etc., within which a computer program is stored, and the computer program, after being loaded into a machine (such as a computer or a smart phone), is used for executing the method for blood pressure measurement previously described.

In FIG. 2, a blood pressure measurement system in accordance with the present invention includes a measuring unit 10 and a computing unit 20 is shown. The measuring unit 10 and the computing unit 20 are connected through a network 30. The measuring unit 10 is, for example, a vibrating pulse sensor of a smartwatch, or an optical sensor of a camera of a smartphone.

The measuring unit 10 is used for measuring a personal electrophysiological signal, such as a pulse signal, the systolic blood pressure, the diastolic blood pressure, or the heart rate. The computing unit 20 is a personal computer (PC) or a cloud server. In one embodiment, the measuring unit 10 has a user interface provided by a software program to allow basic personal information to be input thereon. In an embodiment, the basic personal information includes parameters such as age, height, weight, gender, body fat ratio, and/or data contained in cloud electronic medical records.

The computing unit 20 receives the electrophysiological signals and the basic personal information from the measuring unit 10 via the network 30. The computing unit 20 includes a modeling module 201, an optimization module 202 and a database module 203. The modeling module 201 and the optimization module 202 are software programs written in C, C++, C#, JAVA, Python, or other programming languages supporting network connection function, and the database module 203 is a HIS, NIS , HL7, or a general database (such as MySQL, SQL Server, Oracle, or Microsoft Access). The format in which the measuring unit 10 transmits the electrophysiological signal and the basic personal information to the computing unit 20 may be, but not limited to JSON, XML, YAML or other customized formats.

In one embodiment, the network 30 is, but not limited to a wireless LAN, an infrared or Bluetooth wireless network, or a wired network.

The modeling module 201 establishes a personalized cardiovascular model through a numerical method based on the electrophysiological signal and the basic personal information. In an embodiment, the personalized cardiovascular model has default model parameters, and creates a human physiological parameter. In one embodiment, the numerical method is a finite element method. The details about how to establish a personalized cardiovascular model and obtain a human physiological parameter therefrom have been described above, further description hereby omitted.

The optimization module 202 calibrates the default model parameters through an optimization algorithm and re-establishes the personalized cardiovascular model. In an embodiment, the optimization algorithm is a genetic algorithm, a neural network algorithm, an intelligent algorithm, or any novel algorithm that automatically optimizes and calibrates the default values of the model parameters using a large number of samples, such that the human physiological parameter created by the re-established personalized cardiovascular model matches the measured personal electrophysiological signal.

In one embodiment, the human physiological parameter is the systolic blood pressure, the diastolic blood pressure, or the heart rate. The default model parameters include, but is not limited to vascular wall thickness, vessel diameter, vessel density, blood vessel elasticity, blood concentration, blood density, blood viscosity coefficient, blood flow, blood flow field pressure, muscle elasticity and density, elasticity and density of the bones, skin elasticity or elasticity and density of all the tissues from the wrist continuously extended to the heart.

The database module 203 is used for storing the measured electrophysiological signal, the inputted basic personal information, the established personalized cardiovascular model, and the optimized and re-established personalized cardiovascular model.

The system and method for blood pressure measurement, the computer program product that uses the method, and the computer readable recording medium thereof in accordance with the present invention eliminates the use of the cuff by measuring the personal electrophysiological signal with a measuring unit in the form of a vibrating pulse sensor or an optical sensor. The measuring unit further provides a user interface that facilitates user operations, input of the basic personal information and display of measurement results. The present invention thus eliminates the inconvenience associated with traditional cuff-type blood pressure measuring devices. The present invention also has a smaller volume which makes it easier to carry. The measured personal electrophysiological signal and the inputted basic personal information can be transmitted to a cloud server for integration and operation in order to establish a personalized cardiovascular model, and then the human physiological parameter (i.e., the blood pressure) can be provided to the user based on the modeling results.

In addition, the system for blood pressure measurement in accordance with the present invention can be further combined with the database of a medical center to establish long-range medical care services and early warning systems, and the personalized cardiovascular model is continuously updated by the optimization algorithm to get the latest accurate blood pressure, thereby achieving health management and care anytime and anywhere based on the blood pressure.

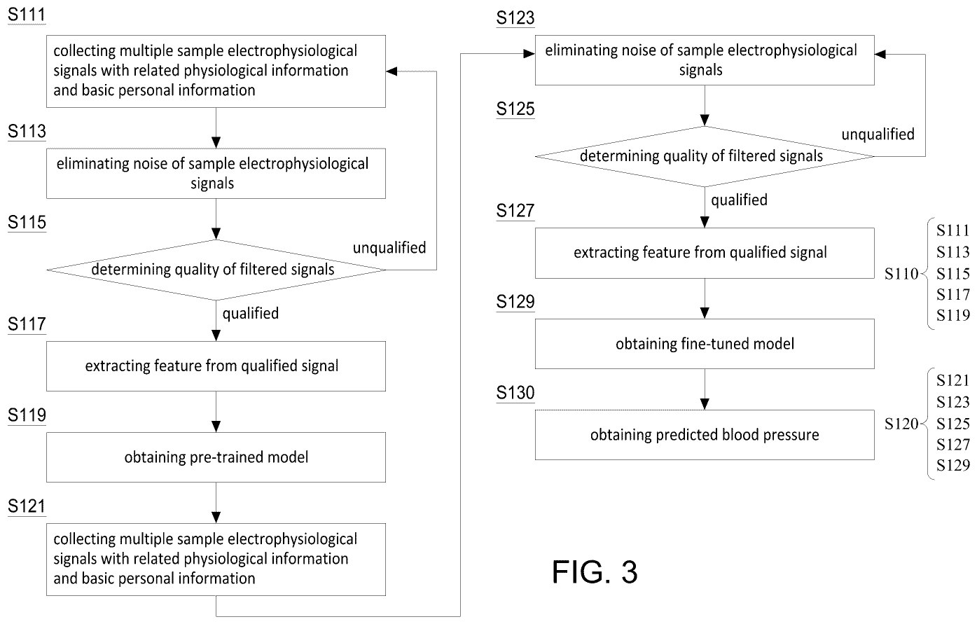

Referring to FIG. 3, a method of measuring blood pressure of a subject (a subject herein is a body with a measurable blood pressure, for example, the subject is a human or an animal.) in accordance with the present invention includes: establishing a pre-trained model (step S110); establishing a fine-tuned model based on the pre-trained model (step S120); and obtaining a predicted blood pressure of the subject by the fine-tuned model (step S130). In one embodiment, the pre-trained model is built via deep learning based on data from multiple persons (samples) in Group A, the fine-tuned model is obtained via transfer learning based on data from multiple persons (samples) in Group B, wherein the characteristics of persons in Group B are more similar to that of the subject than that of persons in Group A. The characteristic types include gender, race, disease type, age range, weight range, height range, whether pregnant, etc. In one embodiment, the subject, and the persons in Group B are pregnant women, but the persons in Group A (generic group) include men and women. Furthermore, the method also can be used for predicting blood pressure for pregnant women by Photoplethysmography (PPG) signal for prognosis of gestational hypertension or preeclampsia via deep learning and personalization.

In details, step S110 further includes: collecting multiple sample electrophysiological signals with related physiological information and basic personal information (step S111); eliminating noise of the sample electrophysiological signals (step S113); determining quality of filtered signals (step S115); extracting feature from qualified signal (step S117); and obtaining the pre-trained model (step S119).

In step S111, acquire multiple sample electrophysiological signals and related basic personal information in Group A. Each sample electrophysiological signal in step S111 is received from a person (sample) in Group A with corresponding physiological information and basic personal information. In one embodiment, the sample electrophysiological signal, the physiological information, and the basic personal information can be obtained through the method mentioned above. In other words, the details of step S111 can refer to that of step S01, wherein the difference between step S10 and step S111 is the data in step S01 is received from the subject (one person) but the data in step S111 is received from multiple persons (samples) in Group A. In other embodiments, the sample electrophysiological signals, the related physiological information, and the related basic personal information are obtained from a database. In one embodiment, the sample electrophysiological signal includes a Photoplethysmography (PPG) signal, and a raw PPG signal can be measured by a PPG sensing patch with 1000 Hz sampling rate. The physiological information includes Systolic Blood Pressure (SBP) and Diastolic Blood Pressure (DBP), and the basic personal information includes gender, height, and weight.

In step S113, a 4th-order Butterworth bandpass filter was utilized to eliminate noise of the raw PPG signal. During data collection, the PPG sensing patch may have received noise from various sources, such as internal components, ambient light, or person movement, wherein the noise will affect signal quality and reduce the accuracy of the deep learning model. Eliminating noise from the raw PPG signal to form a filtered signal can improve the accuracy of subsequent deep learning models. The passband was set to 0.5-5 Hz to retain all peaks and notches of the PPG waveforms while removing high frequency noise and low frequency DC drifting. In other embodiments, the passband range can be modified as 1-4 Hz based on the quality of the raw PPG signals.

In step S115, the filtered PPG signals were segmented into 6-second windows, and each segment was evaluated using a Power Spectral Density (PSD) check to determine its quality. The filtered PPG signals underwent screening based on their PSD, which fast Fourier transform (FFT). In one embodiment, the PSD check was performed using a criterion of 0.15, which implies that the ratio of the harmonics from the PPG peaks must be greater than 15% of the entire sequence. For PPG signals with a PSD value above 0.15 (alleged the qualified signals), the peaks and harmonics of the qualified signals were apparent in both the time domain and frequency domain, indicating that these signals were suitable for the deep learning model.

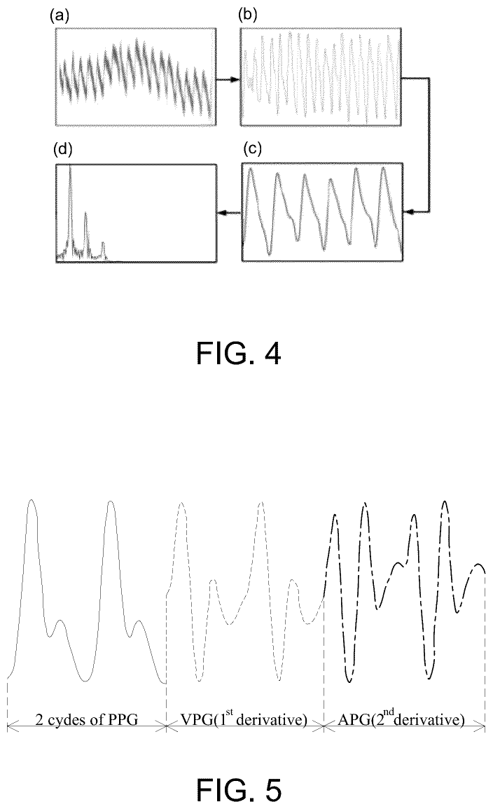

Referring to FIG. 4, FIG. 4 shows (a) a raw PPG signal in step S111; (b) a filtered PPG signal in step S113; and (c) a segment filter PPG signal and (d) a PSD spectrum in step S115.

Referring to FIG. 3 and FIG. 5. In step S117, extracting features from the qualified PPG signals to form characteristic signals, respectively. Each qualified signal was divided into 2-cycle segments, which were resampled to 1000 sample points. The resulting segments, together with their first and second derivatives (also called Velocity of PPG (VPG) and acceleration plethysmogram (APG)), were normalized using min-max normalization as

$${x}_{scaled} = \frac{x - {x}_{min}}{{x}_{max} - {x}_{min}} .$$

Subsequently, the sequence (alleged the characteristic signal) was concatenated into a (1000×3) data for subsequent use.

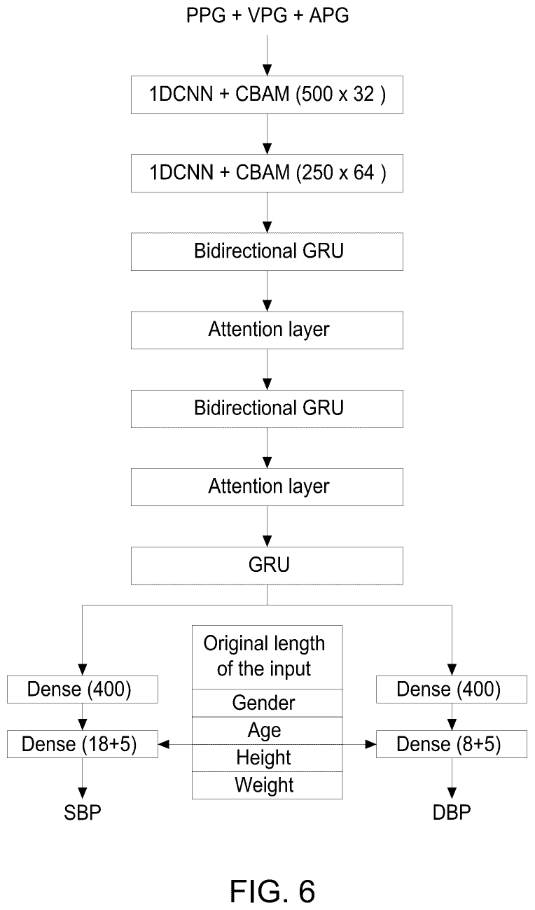

Referring to FIG. 6, FIG. 6 illustrates a model architecture of a proposed deep learning model, wherein the proposed deep learning model can be trained to form the pre-trained model. In step S119, obtaining the pre-trained model based on the characteristic signals (from step S117), related real SBP value, real DBP value, and the basic personal information (from step S111).

The proposed deep learning model consists of four Convolutional Neural Network (CNN) blocks with Channel Attention and Spatial Attention (CBAM) mechanisms for feature extraction and enhancement. Three concatenated attention and bi-directional Gated Recurrent Unit (GRU) layers are utilized to analyze the timing relationship of the extracted features. Two dense layers have been connected separately, where the biological information of the subject (also call the person in Group A in step S119) is incorporated, including gender, age, height, weight, and the original length of the 2-cycle PPG waveform, and the layers eventually output the predicted SBP and DBP values. The CBAM mechanism uses a simpler method to first pool the convolutional output and calculate the spatial/channel weights, thereby enhancing the spatial and channel-wise attention of the model. As PPG signal is a 1D time series, 1D CNN is employed to automatically extract the spatial features of the PPG waveform. In 1D CNN, the machine-trained kernel slides through the input signal in only one direction to perform convolution and extract features.

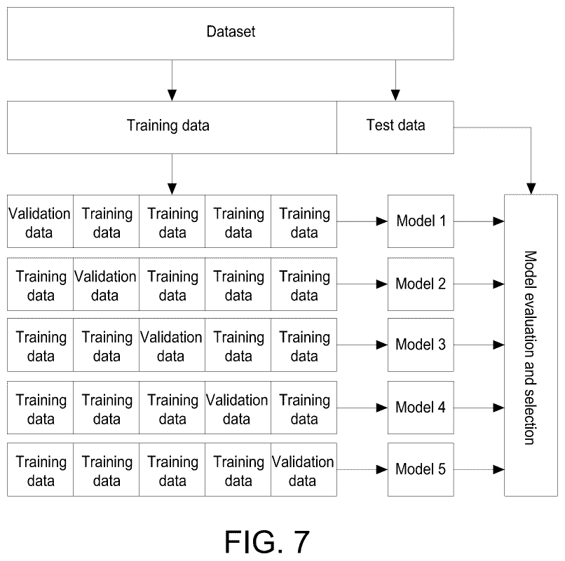

In one embodiment, the dataset of Group A comprised of 456 waveform files, each representing a unique subject. To split the dataset for training the proposed deep learning model, the files were randomly shuffled and divided into two sets, with 85% of the data allocated to the training set and the remaining 15% to the testing set. During the training process, a K-fold cross-validation technique was employed to further prevent overfitting. The K-fold cross-validation approach involves dividing the training set into K equally sized subsets, where K is a hyperparameter that determines the number of times the model is trained and validated. In one embodiment, K was set to 5 as it provided a good balance between preventing overfitting and reducing overall training time, as shown in FIG. 7. The detailed procedure of the 5-fold cross-validation involved training the model for five iterations, in each iteration, one of the subsets was chosen as the validation set while the remaining four were combined to form the training set. Eventually, the testing set was used to evaluate the five models and select the one with the best performance as the pre-trained model.

Optionally, since the prediction of blood pressure value is a regression process, the loss function selected for the deep learning model is Mean Square Error. The equation of mean square error is

$$MSE = \frac{1}{N} {\sum }_{i = 1}^{N} {( {y}_{i} - \hat{y} i )}^{2} .$$

The Adam optimizer was chosen in one embodiment due to the complexity of the proposed model architecture and the larger number of parameters. Typical optimizers like the stochastic gradient descent (SGD) can be slow to converge in such cases. The advantage of using Adam optimizer is that it contains the momentum mechanism, which helps the algorithm converge at a higher speed. Additionally, Adam optimizer has an adaptive learning rate mechanism, which ensures a smooth training process.

Referring to FIG. 3 again. Step S120 further includes: collecting multiple sample electrophysiological signals with related physiological information and basic personal information (step S121); eliminating noise of the sample electrophysiological signals (step S123); determining quality of filtered signals (step S125); extracting feature from qualified signal (step S127); and obtaining the fine-tuned model (step S129).

The difference between step S121 and step S111 is different groups of data sources. In other words, step 121 performs the same collection process as step 111 to collect data from Group B. In one embodiment, the sampling rate in step S121 is lower than that of in step 111, for example, a raw PPG signal is measured with 1000 Hz in step 111, but is measured with 250 Hz in step 121. Further, amount of raw PPG signals collected in step S121 can be less than that of in Step S111. For example, the raw PPG signals in S111 are obtained from 154 normal persons at a sampling rate of 1000 Hz, but the raw PPG signals in step S121 are obtained from 40 pregnant women at a sampling rate of 250 Hz, wherein the subject for predicting a blood pressure is a pregnant woman, and the raw PPG signal of the subject is also measured at a sampling rate of 250 Hz.

In step S123, obtain the filtered PPG signals from the raw PPG signals received by step S121. For the filtering process of step 123, please refer to above step S113. Besides, except that the data sources are different, steps S125 and S127 perform the same processing as steps S115 and S117, respectively. For details of steps S125 and S127, please refer to the relevant paragraphs above.

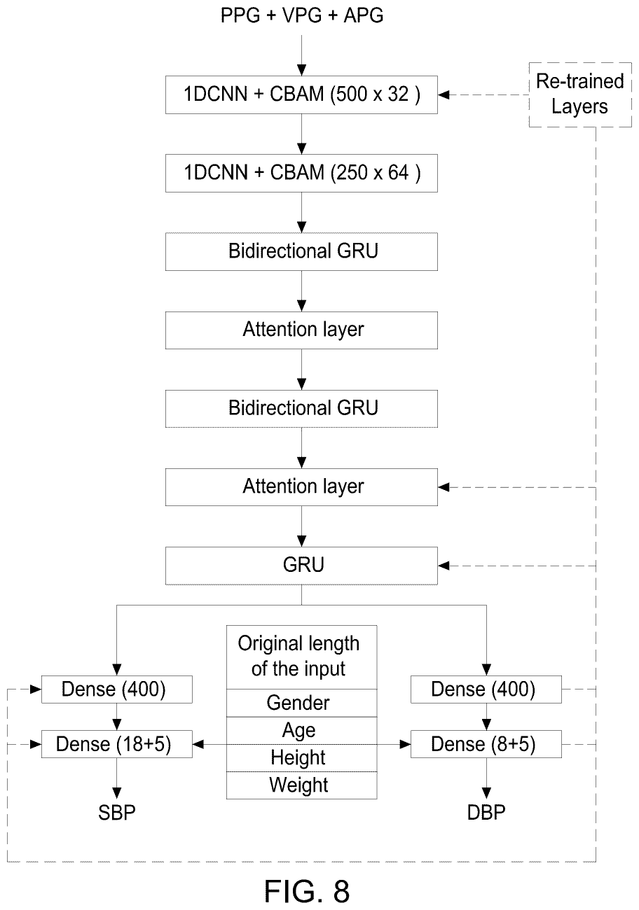

Please refer to FIG. 3 and FIG. 8. In step S129, retrain the pre-trained model based on data from step S121. Transfer learning can be utilized as depicted in FIG. 8, whereby most layers of the pre-trained model are fixed while only a few layers are retrained using the new data. In detail, the second CBAM layer and the following three layers (two Bidirectional GRU lay and one Attention layer) can be fixed, and the other layers are retrained via the new data from Group B to obtain the fine-tuned model. The dataset splitting and training process, please refer to the previous step S119.

Optionally, step S120 can be performed multiple times to obtain a more accurate fine-tuned model. In details, the second step S121 is applied to persons (samples) in Group C to collect data, wherein the characteristics of persons in Group C are more similar to that of the subject than that of persons in Group B. In one embodiment, the persons (samples) in Group B includes the subject. Further, step S120 can be performed again using only data from the subject to obtain a more accurate fine-tuned model (alleged a personalized model). In some embodiments, Group A and Group B both include the subject. In other embodiments, Group A and Group B both exclude the subject.

In step S130, collecting a personal electrophysiological signal and basic personal information of the subject to obtain the predicted blood pressure. The personal electrophysiological signal includes a PPG signal obtained from the subject. Please refer to the above for the signal measurement methods and the data acquisition methods. In one embodiment, the PPG signal undergoes the same processing as steps S113, S115, and S117, and then be input with the basic personal information of the subject to the fine-tuned model to obtain the predicted blood pressure, which includes SBP and DBP values.

In some embodiments, the filtered PPG signals can also be used for biometric identification. A deep learning model combines Convolutional Neural Network (CNN) and Long Short-Term Memory (LSTM) layers can be obtained by the filtered PPG signals for user identification. To train a user identification model, one vs rest strategy was employed. Multiple segments of 6-second PPG signals that have passed quality assessment from each participant are used as training data to train the model. These data were split into an 80% training set and a 20% testing set. Eventually, the developed model can determine whether the user is authenticated based on the 6-second PPG signal input.

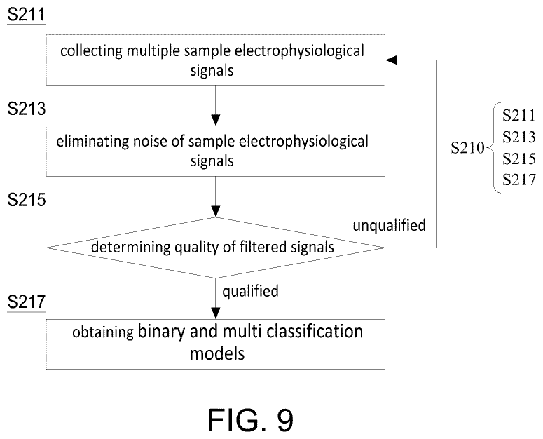

In detail, referring to FIG. 9, a method for biometric identification includes: collecting multiple sample electrophysiological signals (step S211); eliminating noise of the sample electrophysiological signals (step S213); determining quality of filtered signals (step S215); and obtaining the binary and multi classification models (step S217).

In step S211, collect a sample electrophysiological signal from each person to be authenticated. In one embodiment, the sample electrophysiological signals in step S211 includes PPG signals, and each PPG signal contains at least two minutes continuous waveform segment.

In step S213, a 4th-order Butterworth bandpass filter was utilized to eliminate noise of the raw PPG signal. The passband was set to 1-4 Hz to eliminate high-frequency noise and baseline wander, ensuring that the filtered PPG signals maintain their fidelity and relevance for subsequent analysis.

In step S215, each filtered PPG signal undergoes quality assessment algorithm using Power Spectrum Density (PSD) and Direct Current (DC) drift to ensure acquiring a good PPG signal.

First, determine the focus window of the filtered PPG signal. To achieve higher accuracy in biometric identification, we adjust the position of a focus window in each filtered PPG signal. In one embodiment, the window size was fixed to six seconds, and ensure that each window contains a complete PPG signal with complete waveforms from the beginning to the end. If there are incomplete PPG signals, resampling is performed to ensure that the PPG signals have a complete duration of six seconds.

After undergoing a comprehensive filtering and modification process, the PPG signals underwent scrutiny through Power Spectral Density (PSD) analysis. The PSD is derived by squaring the magnitude of the Fourier transform of the signal. Given that the strong correlation between PSD and heart rate, PSD can serve as an indicator to assess whether the acquired PPG signal is attributed to actual heartbeat pulsations. The PSD of the signals can be calculated by

$$PSD = \frac{{\int }_{- ∞}^{∞} {X}^{2} ( ω ) d ω}{{\sum }_{n = 1}^{∞} ( {\int }_{n {ω}_{m} - {ω}_{t}}^{n {ω}_{m} + {ω}_{t}} {X}^{2} ( ω ) d ω )} .$$

In one embodiment, the criterion for PSD checking is set to 0.3, indicating that the proportion of harmonics derived from PPG peaks should exceed 30% of the entire signal sequence.

Following the PSD analysis, a subsequent examination is conducted to assess the presence of DC drift. The acquisition of PPG signals is notably challenged by the occurrence of DC drift, a phenomenon primarily induced by the unintentional movement of the person's hand and the sensor collector during the measurement process. This drift in the direct current (DC) component of the signal poses a substantial obstacle to obtaining accurate and reliable physiological data. The inadvertent shifts in position introduce baseline fluctuations, complicating the interpretation of PPG waveforms and potentially leading to misinterpretation of vital information. Addressing the challenge of DC drift becomes imperative in ensuring the fidelity of acquired PPG signals, thereby enhancing the precision and effectiveness of the physiological monitoring system.

Strategies aimed at mitigating DC drift are essential for fortifying the reliability of PPG signal acquisition in dynamic environments, where patient movement is an inherent aspect of the measurement process, presented as

$${DC}_{drift} = \frac{{V}_{peak ( max )} - {V}_{peak ( min )}}{{\sum }_{n = 1}^{n = peaknumber} \frac{{V}_{peaknumber}}{peaknumber}} ,$$

where Vpeak(max) represents the maximum peak value and Vpeak(min) indicates the minimum peak value. Additionally, peaknumber corresponds to the quantity of peaks within a specified window. In one embodiment, the DC drift check is guided by a criterion set at 0.3. Therefore, the qualified PPG signals can be obtained after step S215.

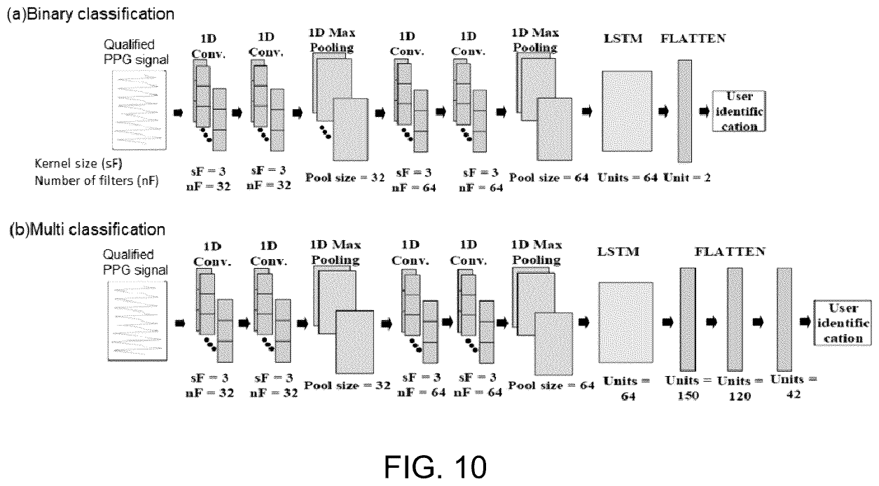

In step S217, obtain two biometric identification models, wherein FIG. 10 shows the model architecture for (a) binary classification and (b) multi classification. As shown in FIG. 10, the binary classification model adopts a model architecture with four 1D convolutional layers, two maximum pooling layers, and one LSTM layer. In detail, after two 1D CNN layers, there are one max pooling layer, two ID CNN layers, one 1D Max pooling layer, and one LTSM layer in sequence. Further, one flatten layer is connect to the LTSM layer in binary classification model.

A kernel size of 3, number of filters of 32 and 64 are used in the binary classification model. The introduction of 64 LSTM units emerges as a pivotal enhancement, profoundly enriching the model's comprehension of temporal dependencies within processed PPG signals. Moreover, the LSTM layer effectively addresses long-time dependencies and the vanishing gradients problem in sequential data. This augmentation significantly fortifies the model's discriminative capabilities, elevating it to the status of a dynamic tool adept at capturing intricate patterns unfolding over time.

Compared with the binary classification model, the multi classification model elevates the architecture with the incorporation of three consecutive flatten layers. Each flatten layer is calibrated with 150, 120, and 42 units, respectively. This deliberate augmentation not only broadens the model's capacity for intricate feature transformation but also amplifies its discernment, improving its adeptness in recognizing and classifying an extensive array of physiological patterns. This augmentation not only broadens the model's capacity for intricate feature transformation but also amplifies its discernment, improving its adeptness in recognizing and classifying an extensive array of physiological patterns. The orchestrated interplay between the 1D convolutional layers and max pooling remains a constant, fortifying the establishment of an efficient hierarchical feature extraction process.

Moreover, in certain scenarios where subject bias can significantly impact the evaluation process, an additional technique known as “leave-one-subject-out cross-validation” is employed. This approach ensures an unbiased evaluation of the model's performance. It achieves this by systematically excluding one subject's data from the training and validation process in each iteration, allowing the model to be assessed in a manner that is less influenced by the specific characteristics of individual subjects. This augmented layer of evaluation serves to enhance the validation of the model's generalization capabilities across a diverse spectrum of subjects or data subsets.

After obtain the binary classification model and the multi classification model, the measured PPG signal can be inputted into above models to confirm whether the PPG signal's owner is an authenticated person. In one embodiment, the measured PPG signal will be preprocessed before being input to the model, wherein the preprocessing process includes noise elimination (step S213) and quality assessment (step S215). In some embodiments, the biometric identification method is suitable for groups with a limited number of users, such as identification of students in a classroom, identification of meeting participants, etc. In one embodiment, the number of users in the group is less than 50 to ensure accuracy.

Optionally, the quality check processes (such as steps S115, S125, S217 above) further include assessing the presence of motion artifact noise. If it is considered that there is no motion artifact noise, the filtered signal then be considered as the qualified signal for subsequent use.

In one embodiment, Kurtosis is employed as the primary criterion to assess the presence of excessive motion artifacts. Kurtosis is a statistical measure that characterizes the tail thickness or peakiness of a probability distribution and is defined as follows:

$$kurtosis = \frac{\frac{1}{n} {\sum }_{1}^{n} {( {X}_{i} - \overline{X} )}^{4}}{{σ}^{4}} ,$$

where Xᵢ represents the sample point values between each peak in the PPG signal, X̅ denotes the mean of X, and σ signifies the variance of the X distribution.

The approach involves calculating the kurtosis value for each data point between every pair of peaks within the PPG signal using above formula. Within the signal segment, the kurtosis values for adjacent peak-to-peak intervals are compared to identify the maximum difference in kurtosis values as the quality check indicator. In one embodiment, the Kurtosis threshold is set at 3.5 to effectively filter out excessive motion artifacts. Subsequently, complete signal segments with a kurtosis value below 3.5 are considered as qualified signals.

The above methods can be all executed at the remote end, all at the edge side, or part of the remote end and part of the edge side. In one embodiment, step 110 and 120 are executed at a cloud server, and step 130 is executed at a device near the user. In addition, data can be stored remotely or pre-stored on chips, devices, and systems near the user. The risk of personal data being compromised can be reduced when all steps are executed offline.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A method of predicting a blood pressure of a subject, comprising:
providing a plurality of first sample electrophysiological signals and a plurality of first measured blood pressures, respectively, wherein each first sample electrophysiological signal of the plurality of first sample electrophysiological signals corresponds to a person in a first group;
creating a plurality of first characteristic signals, wherein each first characteristic signal of the plurality of first characteristic signals consists of a first feature segment extracting from the first sample electrophysiological signal, a first derivative of the first feature segment, and a second derivative of the first feature segment in sequence;
establishing a pre-trained model based on the plurality of first characteristic signals and the plurality of first measured blood pressures;
providing a plurality of second sample electrophysiological signals and a plurality of second measured blood pressures, respectively, wherein each second sample electrophysiological signal of the plurality of second sample electrophysiological signals corresponds to a person in a second group;
establishing a fine-tuned model by retraining the pre-trained model based on the plurality of second sample electrophysiological signals; and
obtaining the blood pressure by inputting a personal electrophysiological signal and a basic personal information from the subject into the fine-tuned model;
wherein, the person in the second group are more similar to the subject in physiological characteristics than the person in the first group.

2. The method of claim 1, wherein the plurality of first sample electrophysiological signals is greater than the plurality of second sample electrophysiological signals in quantity.

3. The method of claim 1, wherein the plurality of first sample electrophysiological signals is greater than the plurality of second sample electrophysiological signals in sampling rate.

4. The method of claim 1, wherein the plurality of second sample electrophysiological signals and the personal electrophysiological signal have the same sampling rate.

5. The method of claim 1, wherein the plurality of first sample electrophysiological signals, the plurality of second sample electrophysiological signals, and the personal electrophysiological signals are Photoplethysmography (PPG) signals.

6. The method of claim 1, further comprising eliminating noise of the plurality of first sample electrophysiological signals before creating the plurality of first characteristic signals.

7. The method of claim 1, further comprising retraining the fine-tuned model based on a subject information received from the subject before obtaining the blood pressure.

8. The method of claim 7, wherein the subject information comprises a measured blood pressure and the subject basic information.

9. The method of claim 1, wherein a first measured blood pressure of the plurality of first measure blood pressure comprises a systolic blood pressure value and a diastolic blood pressure value.

10. The method of claim 1, wherein the subject is a pregnant woman, and the person in the first group excludes any pregnant woman.

11. The method of claim 1, further comprising using the blood pressure to identify gestational hypertension or preeclampsia.

12. The method of claim 1, wherein the fine-tuned model is established based on a plurality of second characteristic signals, wherein each second characteristic signal of the plurality of second characteristic signals consists of a second feature segment extracting from the second sample electrophysiological signal, a first derivative of the second feature segment, and a second derivative of the first second segment in sequence.

13. The method of claim 1, wherein the basic personal information comprises gender, age, height, and weight.

14. The method of claim 1, wherein the pre-trained model is established based on the basic personal information from the person in the first group.

15. The method of claim 1, wherein the fine-tuned model is established based on the basic personal information from the person in the second group.

16. The method of claim 1, wherein the first feature segment is two complete continuous waveforms in the first sample electrophysiological signal.

17. The method of claim 1, wherein the personal electrophysiological signal is transferred to a subject characteristic signal before inputting into the fine-tuned model, and the subject characteristic signal consists of a subject feature segment extracting from the personal electrophysiological signal, a first derivative of the subject feature segment, and a second derivative of the subject feature segment in sequence.
